(19) United States
(12) Patent Application Publication
(10) Pub. No.: US 2026/0086015 A1
(43) Pub. Date: Mar. 26, 2026

(54) SYSTEMS AND PROCESSES FOR MONITORING LIQUID CONDENSATE AND NATURAL GAS LIQUIDS

(71) Applicant: SAUDI ARABIAN OIL COMPANY, Dhahran (SA)

(72) Inventors: Maha NOUR, Dhahran (SA); Khawlah KHARASHI, Dhahran (SA); Diki ANDRIAN, Dhahran (SA); Khalid HAZAZI, Dhahran (SA); Ali ALDOSSARY, Dhahran (SA)

(73) Assignee: SAUDI ARABIAN OIL COMPANY, Dhahran (SA)

(21) Appl. No.: 18/895,034

(22) Filed: Sep. 24, 2024

(51) Int. Cl. G01N 15/075; G01N 15/00; G01N 33/28

(52) U.S. Cl. CPC G01N 15/075; G01N 2015/003; G01N 33/2847

(57) ABSTRACT

The present disclosure provides systems and methods for monitoring liquid condensate and natural gas liquids (NGL) in an inflow stream. Methods for detecting water content may include capturing one or more images of a flow stream, the flow stream comprising a water component and at least one of a liquid condensate component and a natural gas liquid component, sensing one or more bubble images captured within the one or more images of the flow stream, the one or more bubble images representing one or more water bubbles within the flow stream, constructing one or more reference geometries based on the one or more bubble images, each of the one or more reference geometries having a parameter associated with each of the one or bubble images, and based on the parameter for each of the one or bubble images, producing a predicted water content value for the flow stream.

### FIELD OF THE DISCLOSURE

The present disclosure relates generally to natural gas processing and, more particularly, to utilizing a charged-coupled device to monitor liquid condensate and natural gas liquids (NGL) in an inflow stream.

### BACKGROUND OF THE DISCLOSURE

Reliably measuring the water concentration in oil and gas industries is critical for operational safety, corrosion avoidance, and quality product control. The current state of the art provides solutions related to water content measurement in hydrocarbon components for upstream, midstream, and downstream facilities. However, there are limited technologies available for highly sensitive water detection in liquid condensate and NGL flow stream applications, especially in range of about 0 parts per million (ppm) or more to about 3000 ppm or less. This may present quality control and efficiency challenges where undetected water in a liquid condensate or NGL flow stream causes system delays resulting from corrosion, quality deficit, and the like.

Although current techniques for liquid condensate and NGL flow stream assessment, and for monitoring free water concentration in liquid condensate and NGLs in particular, are based on technological advancements made over many years, current assessment techniques may still be ineffective to achieve ideal results. For example, control and monitoring systems for liquid condensate and NGL assessment may be unreliable and inefficient. Accordingly, there is an impetus to improve liquid condensate and NGL assessment technology to overcome current technological challenges by implementing improvements including, for example: enhancing the control and monitoring systems within a liquid condensate and NGL assessment system, reducing inefficiencies associated with liquid condensate and NGL monitoring systems, increasing the throughput quality of liquid condensate and NGL processing systems, reducing errors associated with current control and monitoring systems, decreasing the cost of liquid condensate and NGL processing, and the like.

Consequently, there exists a need for further improvements in liquid condensate and NGL processing technology to overcome the aforementioned technical challenges and other challenges not mentioned.

### SUMMARY OF THE DISCLOSURE

Various details of the present disclosure are hereinafter summarized to provide a basic understanding. This summary is not an exhaustive overview of the disclosure and is neither intended to identify certain elements of the disclosure, nor to delineate the scope thereof. Rather, the primary purpose of this summary is to present some concepts of the disclosure in a simplified form prior to the more detailed description that is presented hereinafter.

According to an embodiment consistent with the present disclosure, a method for detecting water content may include one or more steps. The one or more steps may include capturing one or more images of a flow stream, the flow stream comprising a water component and at least one of a liquid condensate component and a natural gas liquid component. The one or more steps may include sensing one or more bubble images captured within the one or more images of the flow stream, the one or more bubble images representing one or more water bubbles within the flow stream, wherein the sensing is refined based on a measured free water content value and the measured free water content value is based on a total water content value and a dissolved water content value. The one or more steps may include constructing one or more reference geometries based on the one or more bubble images, each of the one or more reference geometries having a parameter associated with each of the one or bubble images. The one or more steps may include based on the parameter for each of the one or bubble images, producing a predicted water content value for the flow stream.

In another embodiment, a monitoring system for detecting water content may include a charged couple device (CCD) configured to capture one or more images of a flow stream, the flow stream comprising a water component and at least one of a liquid condensate component and a natural gas liquid component, and a control device having a memory coupled to one or more processors, the one or more processors configured to cause the control device to perform one or more steps. The one or more steps may include capturing one or more images of a flow stream, the flow stream comprising a water component and at least one of a liquid condensate component and a natural gas liquid component. The one or more steps may include sensing one or more bubble images captured within the one or more images of the flow stream, the one or more bubble images representing one or more water bubbles within the flow stream, wherein the sensing is refined based on a measured free water content value and the measured free water content value is based on a total water content value and a dissolved water content value. The one or more steps may include constructing one or more reference geometries based on the one or more bubble images, each of the one or more reference geometries having a parameter associated with each of the one or bubble images. The one or more steps may include based on the parameter for each of the one or bubble images, producing a predicted water content value for the flow stream.

Any combinations of the various embodiments and implementations disclosed herein can be used in a further embodiment, consistent with the disclosure. These and other aspects and features can be appreciated from the following description of certain embodiments presented herein in accordance with the disclosure and the accompanying drawings and claims.

### BRIEF DESCRIPTION OF THE DRAWINGS

FIG. 1 depicts a flow diagram for an example process for implementing a charged-coupled device (CCD) camera as part of a system to capture a free water content value from a natural gas processing inflow stream.

FIG. 2 is a diagram of an example control loop, implemented according to at least one embodiment of the present disclosure.

FIG. 3 is a set of diagrams illustrating the behavior of water, solids, and gas in an aqueous stream.

FIG. 4A is a graph illustrating example water concentration, according to at least one embodiment of the present disclosure.

FIG. 4B is a graph illustrating example predicted water bubble size, according to at least one embodiment of the present disclosure.

FIG. 5 depicts a schematic diagram of an example system, according to at least one embodiment of the present disclosure.

FIG. 6 is a schematic flowchart of an example method for monitoring liquid condensate and natural gas liquids (NGLs) in an inflow stream, according to at least one embodiment of the present disclosure.

FIG. 7 is an example of a block diagram of a computer system, according to at least one embodiment of the present disclosure.

FIG. 8 depicts a cloud computing environment that can be used to perform one or more actions according to an aspect of the present disclosure.

FIG. 9 is a block diagram of a computer system that may be used to implement one or more of the systems or methods described herein in accordance with certain embodiments.

### DETAILED DESCRIPTION

Embodiments of the present disclosure will now be described in detail with reference to the accompanying drawing figures. Like elements in the various figures may be denoted by like reference numerals. Further, in the following detailed description, specific details are set forth in order to provide a more thorough understanding of the claimed subject matter. However, it will be apparent to one of ordinary skill in the art that the embodiments disclosed herein may be practiced without these specific details, or with details that are not described herein in the interest of clarity. Thus, in some instances, well-known features have not been described in detail to avoid unnecessarily complicating the description. Additionally, it will be apparent to one of ordinary skill in the art that the scale of the elements presented in the accompanying drawing figures may vary without departing from the scope of the present disclosure.

Embodiments in accordance with the present disclosure generally relate to utilizing a charged-coupled device to monitor liquid condensate and natural gas liquids (NGL) in an aqueous stream.

Reliably measuring the water concentration in oil and gas industries is critical for operational safety, corrosion avoidance, and quality product control. Specifically, excessive free water in liquid condensate and NGL may cause system instability, affect the NGL quality, and promote corrosion. Excessive free water may also impact downstream facilities to cause pump seal damage, reboiler fouling, system slippage, and overall energy loss. The current state of the art provides solutions related to water content measurement in hydrocarbon components for upstream and downstream facilities. However, there are limited technologies available for rapid and highly sensitive water detection in liquid condensate and NGL applications, especially in range of about 0 parts per million (ppm) or more to about 3000 ppm or less of excess free water. In many cases, producing liquid condensate and/or NGL includes exposing various hydrocarbons with dissimilar boiling points to elevated pressure to convert natural gas into a liquid phase. This may present repeatability and optimization challenges, specifically where targeted liquid condensate and/or NGL within an inflow stream includes different hydrocarbons with dissimilar boiling points that range from about −100° Fahrenheit (F) or more to about 200° F. or less, although other values are contemplated (e.g., −98° F. to 168° F.). An inflow stream is a component of a natural gas processing stream, where liquid condensate and/or NGLs flow in an aqueous state into a processing chamber for particular natural gas processing operations.

Boiling points and/or condensation points within an inflow solution of targeted liquid condensate and/or targeted NGL may depend on each cut point of hydrocarbon components present in the inflow stream. For example, natural gas may be converted, on account of a high pressure environment, into a liquid phase at about 170° F. or more to about 190° F. or less, although other values are contemplated (e.g., 180° F.). The high-pressure environment may be between 350 pounds per square inch gauge (PSIG) or more to about 450 PSIG or less, although other values are contemplated (e.g., 400 PSIG). Removing excess free water from liquid condensate and/or NGL thus presents a complex challenge, especially where sampling and/or artificially rendering a complex pressure and temperature system is not viable. Accordingly, embodiments of the present disclosure provide a process solution that utilizes an enhanced charged couple device (CCD) camera for detecting unwanted water, gas, and/or solid concentrations in a complex inflow stream in order to capture and characterize liquid condensate and NGL that may be associated with variable hydrocarbon components.

CCD cameras are digital imaging devices that convert light into electronic signals by capturing light within individual and adjacent pixels (i.e., image sensors, photodiodes). The pixels may be disposed on a wafer (e.g., a silicon wafer). Pixels may be tightly-packed and each pixel may be arranged or otherwise configured to capture photons and convert them into a build-up of electrical charge. The amount of charge is proportional to the intensity of light that contacts each pixel. In some cases, the surface of a CCD may be treated with anti-reflective coatings to maximize photon absorption and minimize losses. When photons strike the CCD pixels during exposure to light, the photons generate electron-hole pairs. At each pixel, the electrons may be collected in a potential well, which may be created by applying a voltage to electrodes above the silicon wafer. The amount of charge each pixel can hold (e.g., “well-depth”) may vary by design and affect the dynamic range of the CCD camera. In some cases, CCD cameras read the accumulated charge via charge transfer. Charge transfer includes a vertical shift followed sequentially by a horizontal shift. For example, the charges are transferred between pixels by adjusting the voltages of the electrodes in a sequence, pushing the charge along a row and then down a column to a readout register. At the end of the charge transfer, the charges reach an amplifier where they are converted from an analog signal (e.g., the accumulated charge) to a digital signal through an analog-to-digital converter (ADC). This ADC conversion allows the CCD camera and/or scientific instrument to process and store an image from which the exposed light emanates. CCDs may be optimized to capture images at a “high-resolution”, where a high-resolution may be any resolution sufficient for extracting information relevant to constructing a free water content volume value based on certain characteristics of an image (e.g., about 1μ or less, though other values are contemplated).

As discussed above, there are limited technologies available for highly sensitive water detection in liquid condensate and NGL applications. There exists an impetus to develop inflow monitoring solutions for detecting the water concentration in the ppm range at a liquid phase. Accordingly, embodiments of the present disclosure may utilize a precise CCD camera to detect a free water value that may allow for robust and sensitive water detection for inflow monitoring of liquid condensate and NGL. Specifically, aspects presented herein provide a robust procedure for processing images from a CCD camera based on enhanced prediction techniques informed by iterative coulometric Karl Fischer (KF) titration. In at least one embodiment, embodiments described herein provide a methodology for enhancing water concentration value using imaged data obtained from the CCD camera via real-time or iterative comparison of the enhanced water concentration value with a comparable coulometric KF titration for the inflow stream.

In the current state of the art, coulometric KF titration is a laboratory solution capable of measuring the total water in a ppm range for a volume of gas and/or liquid based on chemical reactions within the gas and/or liquid. Coulometric KF titration may exhibit higher-than-average accuracy. For instance, unlike other titration techniques, a coulometric KF titration can trace low levels of free, emulsified, and dissolved water. When used correctly, the test is capable of measuring water levels as low as about 1 ppm or about 0.0001% concentration. In a typical coulometric KF titration, an iodine reagent is generated electrolytically at an anode within a titration cell. Water content is then determined by measuring the amount of electricity required to produce enough iodine to react with all the water in a sample.

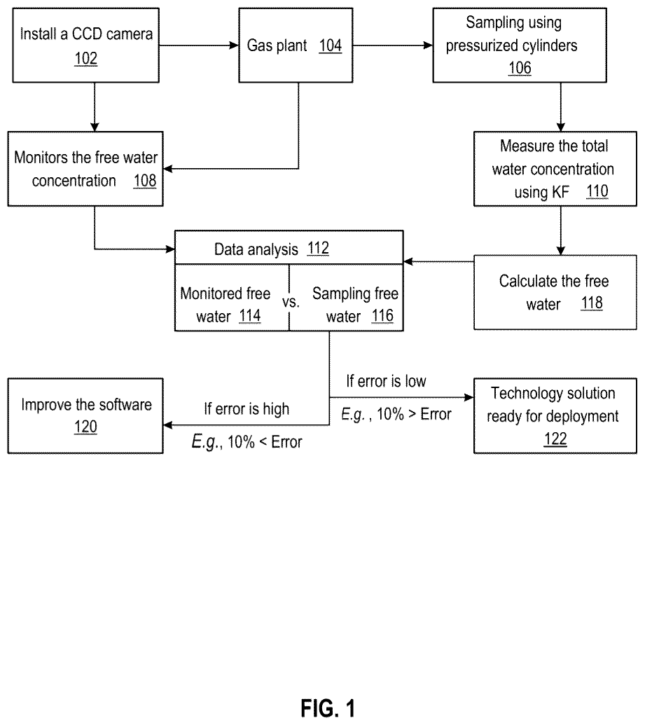

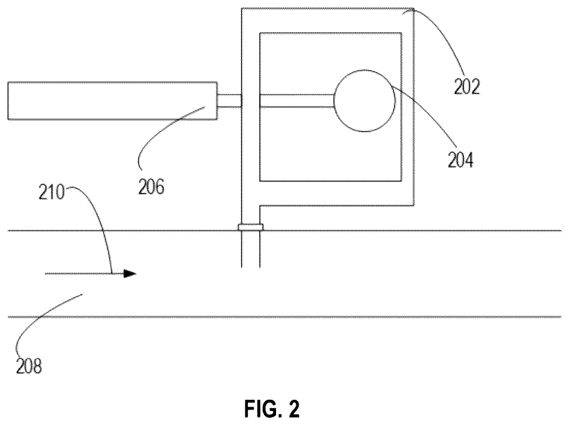

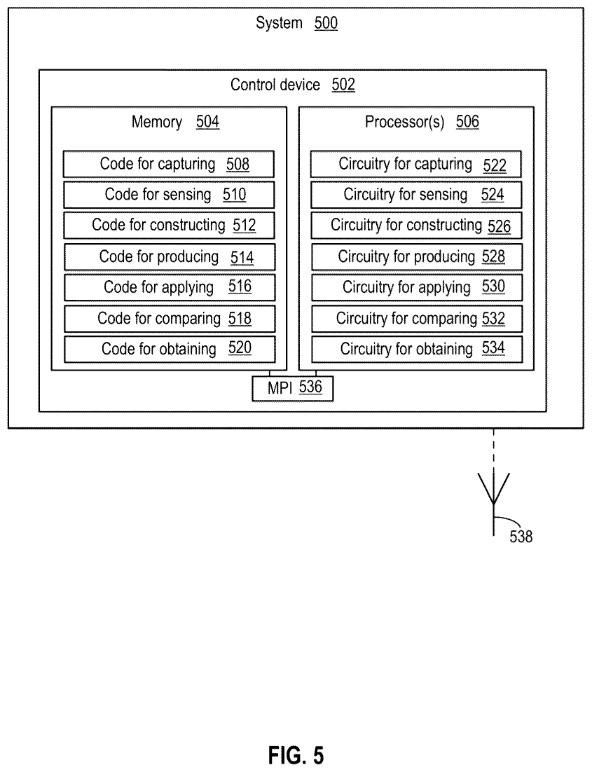

Aspects of the present disclosure provide systems and devices for utilizing a CCD camera to monitor liquid condensate and NGLs in an inflow stream by generating an enhanced water concentration value using imaged data of the inflow stream obtained from the CCD camera. FIG. 1 depicts a flow diagram for an example process for implementing a CCD as part of a system to capture a free water content value from a natural gas processing inflow stream. FIG. 1 depicts a series of steps 102-122, each of which may be performed at a natural gas processing facility (e.g., an upstream facility, a midstream facility, a downstream facility, or some combination therein). The steps 102-122 may be performed in any order, and may be performed in sequence, in parallel, or both. Each of the steps may be performed by a controller or a control device (e.g., the control device 200), the control device arranged or substantially configured to operate elements described herein and to communicate with a computer system (e.g., the computer system 900) via one or more wired or wireless communication lines. The control device (e.g., a control loop) may be implemented at a single location or at multiple locations and may be a supervisory controller or may be in communication with a supervisory controller by way of a communication line. In at least one embodiment, the communication line may be a wireless communication line, a wired communication line, or both, although other types of communication line are contemplated. The control device may include one or more processors coupled to a computer readable medium/memory and configured or substantially arranged to perform certain steps as described here. The control device may be further understood with reference to FIGS. 2A-2B and FIG. 5.

At step 102, a CCD camera is installed at an inflow stream. In one example, a CCD camera may be installed above or directly above a surface of an inflow stream, near or at the surface of the inflow stream, or beneath or directly beneath an inflow stream. Additionally, the camera may be installed adjacent to, directly adjacent to, partially adjacent to, or directly over, at, or under the surface of the inflow stream. The installation system of the CCD camera may include a wired or wireless connection to a control device (e.g., the control device 200), the control device arranged or substantially configured to operate the CCD camera and communicate with a computer system (e.g., the computer system 900) via one or more wired or wireless communication lines as described above. The installation system may include a power supply coupled to the CCD camera and/or the computer system, the power supply configured or substantially arranged to provide power to the CCD camera, the control device, or both.

At step 104, a CCD camera is affixed within a gas plant. The gas plant may be understood to be any gas plant capable of processing natural gas. In one example, step 104 may be similar and comparable to step 102 and may include the same installation system or a distinct installation system. In at least one embodiment, the gas plant may include a light source coupled to a power source, which may be a power source similar to the power source described with respect to step 102. The light source may be arranged or otherwise configured to direct light towards an inflow stream, which may enhance images captured by the CCD camera. In at least one embodiment, the wavelength captured by the CCD camera is within the range of visible light (about 380 nm or more to about 750 nm or less). In at least one embodiment, the CCD camera may be affixed in the gas plant using an inflow loop system on a pipe to monitor the free water in the flowing liquid condensate or NGL. In at least one embodiment, the inflow loop system may be a bypass loop system and may allow for either permanent or transportable installation of the installation system. In at least one embodiment, the pipe may have a diameter of about 0.2 inches or more to about 1.8 inches or less, such as 1 inch, 1.5 inches, 0.375 inches, and the like, although other values are contemplated.

In at least one embodiment, the pipe may be installed vertically. In at least one embodiment, the pipe may be installed horizontally. In one example, the CCD camera may be installed to capture images of an inflow stream moving from a bottom position to top position. This may better capture images of a homogeneous solution within the inflow stream and may provide better measurements useful to construct results for a KF titration.

FIG. 2 is a diagram further illustrating an example of the inflow loop system. The inflow loop system of FIG. 2 includes a flow pipe 202 mounted between a short loop sampler 206 and a CCD camera 204. The inflow loop system of FIG. 2 further includes a main gas plant pipeline 208 configured or otherwise arranged to direct an inflow stream in a flow direction 210. As in FIG. 2A, the pipe 202 is configured or otherwise arranged to extract fluid flow from a main gas plant pipeline 208. The pipe 202 is configured or otherwise arranged to direct fluid flow sampled from the main gas plant pipeline between the short loop sampler 204 and the CCD camera 206.

At step 108, the CCD camera monitors the free water concentration of the liquid condensate and/or NGL. In at least one embodiment, the free water concentration may be the concentration of water detected within a volume of the liquid condensate and/or NGL. In at least one embodiment, the CCD camera monitors the free water concentration within the inflow stream by capturing image information about the inflow stream (e.g., optically captured images, photographs, and the like) and outputting the images to the control device. In at least one embodiment, the CCD camera captures image information with resolution of up to about 1 μm. Generally, the CCD camera is configured or otherwise arranged to capture images on a microscale. Each pixel of an installed CCD camera may have a conversion metric relating to the resolution of the images. For example, about two pixels may cover about 1 μm. In at least one embodiment, the control device causes the CCD to begin monitoring based on an activation signal. The activation signal may be obtained by the CCD camera to activate (i.e., turn on) the CCD camera and begin the monitoring.

At step 106, which may be performed concurrently with or in sequence with above step 108, one or more sampling points along the inflow stream are identified and are configured or otherwise arranged for laboratory analysis via coulometric KF titration. Sampling at one or more sampling points may be performed using liquid pressurized cylinders configured or otherwise arranged to operate at high pressures (about 400 PSIG or more). The pressurized cylinders may capture a sample from the inflow stream and then may be sent for laboratory analysis to measure the total water within the sample. In at least one embodiment, the total water concentration is measured using coulometric KF titration or another known American Society for Testing and Materials (ASTM) method. The pressurized cylinders may pass through fluid sample restoration upon arrival to disperse and/or dissolve any particles that may have formed during sample cooling to ambient conditions. The restoration process may restore the sample to single phase conditions. Following the sample restoration, compositional analysis is performed on the sample using a flash procedure. The flash procedure may provide the hydrocarbon composition, the gas-to-liquid ratio, and the like. Finally, at step 110, the total water concentration within the sample is measured through vapor analysis and through coulometric KF titration. In at least one embodiment, the amount of anhydrous solvents may be measured to determine the micrograms of water-per-gram of solvent, which may facilitate robust correction of a water content value in both gas and liquid compositions.

At step 118, a measured free water content value is calculated based on the total water content value and dissolved water content value determined in steps 106 and 110. In at least one embodiment, the dissolved water value may be equal to the saturation level on account of the existence of the free water. The saturation level is constant for a known medium, as shown in equation (1).

$$K = \frac{{C}_{mix}}{{VP}_{Sat}} \qquad Equation ( 1 )$$

VP_{Sat} may be the saturation vapor of water measurement at the measurement temperature, and Cₘᵢₓ may be a saturation concentration of a hydrocarbon liquid mixture.

In a liquid condensate, there may be a mixture of different hydrocarbons that have different saturation limits. Therefore, in a mixture of hydrocarbons, the saturation level of the mixture can be calculated through knowing the concentration of each component of the medium, as shown in equation (2).

$${C}_{mix} = {\sum }_{i}^{n} {x}_{i} {C}_{i} \qquad Equation ( 2 )$$

xᵢ may be the mass fraction of component in a mixture. Cᵢ may be the saturation concentration of that component (water saturation constant at pure hydrocarbon).

Table 1 below, illustrates examples of the different hydrocarbons available in the liquid condensate.

TABLE 1

| | Mole Fractions | % |
|---|---|---|
| Propane | 0.41497834 | 41.4978340% |
| n-Butane | 0.26170083 | 26.1700830% |
| n-Pentane | 0.11734100 | 11.7341000% |
| i-Pentane | 0.09446531 | 9.4465310% |
| i-Butane | 0.08160902 | 8.1609020% |
| n-Hexane | 0.02152735 | 2.1527350% |
| Ethane | 0.00461257 | 0.4612570% |
| n-Heptane | 0.00209465 | 0.2094650% |
| n-Octane | 0.00116653 | 0.1166531% |
| H₂S | 0.00000562 | 0.0005620% |

In at least one embodiment, the measured free water content may be calculated by subtracting the calculated saturation value of the condensate mixture from the total measured water value. The result of the free water calculation (i.e., the measured free water content value) may be output to the control device discussed above.

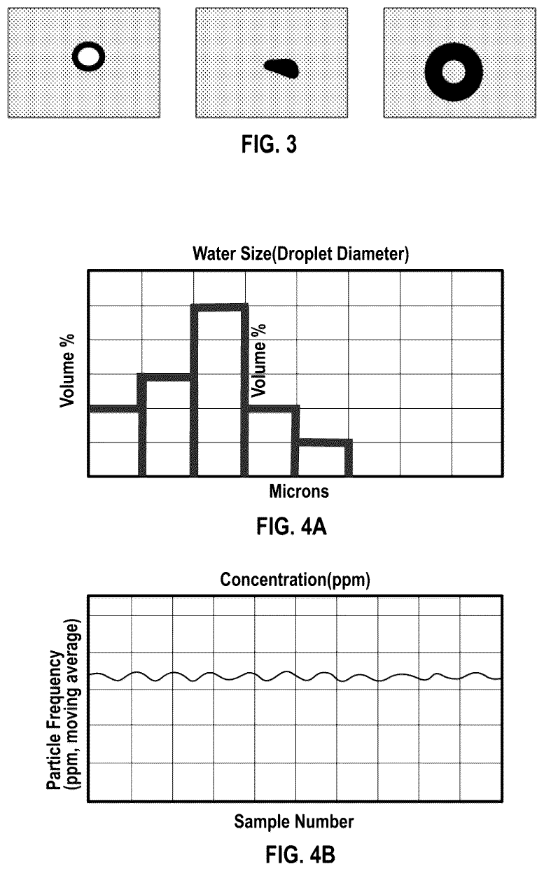

At step 112, the one or more processors of the control device may obtain the captured image information from the CCD camera and may obtain the measured free water value resulting from steps 106, 108 and 118. The one or more processors may obtain the captured image information from the CCD camera via one or more communication lines and may obtain the measured free water content value via one or more communication lines, via direct user input, or via the like. The one or more processors then may begin data analysis and/or image processing operations. Data analysis by the one or more processors proceeds first by implementing a monitored free water step 114. During step 114, a predicted free water content value is detected within the condensate/NGL flow by detecting water bubbles within captured image data and constructing one or more reference geometries based on the one or more bubble images. In at least one embodiment, a reference geometry associated with a bubble image may include a two-dimensional shape and/or a three-dimensional shape. In at least one embodiment, the one or more reference geometries may be explicitly constructed from and separate from the bubble image and may have a superpositional relationship with the bubble image. For example, the reference geometry may be a shape file constructed separate from the bubble image file and superimposed over the bubble image file. In at least one embodiment, the one or more reference geometries may be implicitly constructed from the bubble image and may be an inferred characteristic of the bubble image itself. For example, the reference geometry may be determined as a quality of the bubble image file. In at least one embodiment, the detection is performed using applicable machine learning techniques, such as those described below. Machine learning techniques may be applied to identify and differentiate/correlate water droplets from solid particles and gas bubbles detected within the inflow stream. Detecting the water bubbles may be a direct measurement. As illustrated from left to right in FIG. 3, the water bubbles may have spherical shape, solids in the fluid stream may have random shape, and gas in the fluid stream may have doughnut shapes. The methods and systems provided herein detect such differences and may distinguish between shapes to determine and characterize water bubbles.

In at least one non-limiting example, the one or more reference geometries may be one or more circles disposed over the captured image data to represent each detected water bubble. Based on the diameter of the circles, or based on other parameters of each of the reference geometries, the one or more processors calculate a volume corresponding to a predicted free water content value. Calculating a volume in this manner may include calculating the diameter of the bubbles based on pixel size within each frame of the images obtained from the CCD camera. In certain cases, step 114 measures free and emulsified water, but not dissolved water, to produce the predicted free water content value. Data analysis by the one or more processors then proceeds by implementing a sampling free water step 116. During step 116, the one or more processors compare the measured free water content value with the predicted free water content value. In at least one embodiment, the comparison may be based on calculating the error of the measured free water and the reference free water if the error. The one or more processors then calculate an actual error value (e.g., an error percentage value) based on the comparison. If the actual error value is below an acceptable error value (e.g., about 5% or more to about 15% or less, such as about 10%, though other values are contemplated), then the processors proceed to step 122, implementing the installed CCD camera for monitoring of an inflow stream. If the actual error value is above the acceptable error value, then the processors proceed to step 120, implementing an improvement to the procedure for collecting image data. The improvement to the procedure for collecting image data might include applying a computer vision image processing procedure, applying a time-based machine learning feature (e.g., to observe fluid dynamics over time and detect particles shape/size to quantify the concentration in real time), and the like.

FIGS. 4A and 4B illustrate example graphs describing output from the systems and methods described herein. In the top graph, microns are plotted along the x-axis and volume percentage is plotted on the y-axis to illustrate overall water bubble size within a sample of the inflow stream. In the bottom graph, sample number is plotted along the x-axis and particle frequency (e.g., ppm, moving average) is plotted on the y-axis to illustrate overall concentration of water within the inflow stream over time. In combination, information provided in the example graphs may provide useful insight regarding volume of water within an inflow stream over time, which may allow for adjustment of the gas plant to better meet inflow quality specifications.

Embodiments of steps 114 and 120 described herein can constitute one or more technical improvements over conventional typical CCD camera image capture operations and image processing techniques by tailoring the parameters (e.g., layers, weights as described below) of the operations to more accurately predict free water content within an inflow stream. Additionally, one or more embodiments described herein can have a practical application by training machine learning models to achieve defined natural gas processing efficiency objectives. For example, one or more embodiments described herein can perform accurate, real-time free water content prediction operations.

As used herein, the term “machine learning” can refer to an application of artificial intelligence technologies to automatically and/or autonomously learn and/or improve from an experience (e.g., training data) without explicit programming of the lesson learned and/or improved upon. Machine learning as used herein can include, but is not limited to, deep learning techniques. Various system components described herein can utilize machine learning (e.g., via supervised, unsupervised, and/or reinforcement learning techniques) to perform tasks such as classification, regression, and/or clustering. Execution of machine learning tasks can be facilitated by one or more machine learning models trained on one or more training datasets in accordance with one or more model configuration settings.

As used herein, the term “machine learning model” can refer to a computer model used to facilitate one or more machine learning tasks (e.g., regression and/or classification tasks). For example, a machine learning model can represent relationships (e.g., causal or correlation relationships) between parameters and/or outcomes within the context of a specified domain. For instance, machine learning models can represent the relationships via probabilistic determinations that can be adjusted, updated, and/or redefined based on historic data and/or previous executions of a machine learning task. In various embodiments described herein, machine learning models can simulate a number of interconnected processing units that can resemble abstract versions of neurons. For example, the processing units can be arranged in a plurality of layers (e.g., one or more input layers, hidden layers, and/or output layers) connected by varying connection strengths (e.g., which can be commonly referred to within the art as “weights”).

Machine learning models can learn through training with one or more training datasets; where data with known outcomes in inputted into the machine learning model, outputs regarding the data are compared to the known outcomes, and/or the weights of the machine learning model are autonomously adjusted based on the comparison to replicate the known outcomes. As the one or more machine learning models train (e.g., utilize more training data), the machine learning models can become increasingly accurate; thus, trained machine learning models can accurately analyze data with unknown outcomes, based on lessons learned from training data and/or previous executions, to facilitate one or more machine learning tasks.

Example types of machine learning models can include, but are not limited to: artificial neural network (“ANN”) models, perceptron (“P”) models, feed forward (“FF”) models, radial basis network (“RBF”) models, deep feed forward (“DFF”) models, recurrent neural network (“RNN”) models, long/short memory (“LSTM”) models, gated recurrent unit (“GRU”) models, auto encoder (“AE”) models, variational AE (“VAE”) models, denoising AE (“DAE”) models, sparse AE (“SAE”) models, markov chain (“MC”) models, Hopfield network (“HN”) models, Boltzmann machine (“BM”) models, deep belief network (“DBN”) models, convolutional neural network (“CNN”) models, deep convolutional network (“DCN”) models, deconvolutional network (“DN”) models, deep convolutional inverse graphics network (“DCIGN”) models, generative adversarial network (“GAN”) models, liquid state machine (“LSM”) models, extreme learning machine (“ELM”) models, echo state network (“ESN”) models, deep residual network (“DRN”) models, kohonen network (“KN”) models, support vector machine (“SVM”) models, and/or neural turing machine (“NTM”) models.

FIG. 5 illustrates a schematic diagram of an example system 500, which includes a control device 502. The system 500 may be implemented as part of the installation system described with respect to FIG. 1. The control device 502 may be implemented at a single location or at multiple locations and may be a supervisory controller or may be in communication with a supervisory controller by way of a communication line. In at least one embodiment, the communication line may be a wireless communication line, a wired communication line, or both, though other types of communication line are contemplated.

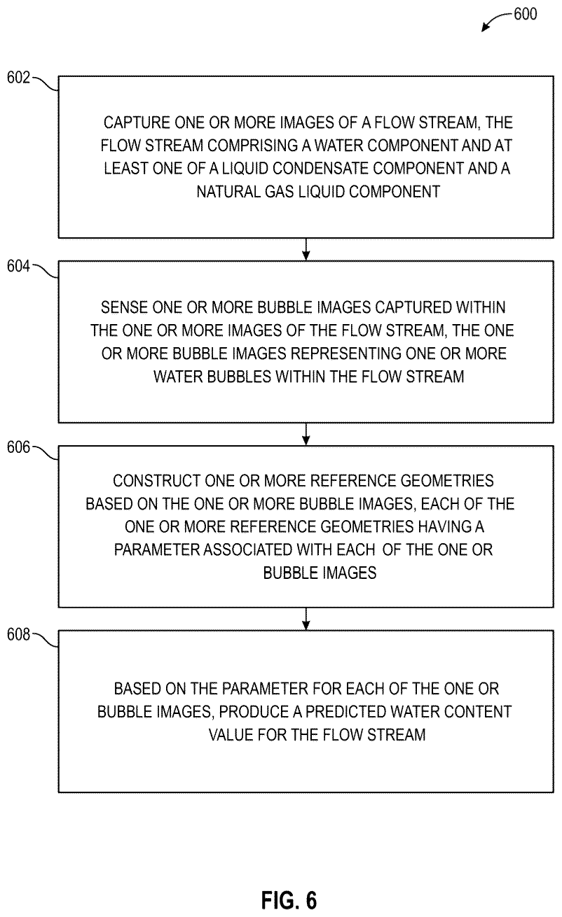

The control device 502 may include a CPU processing system, which may be configured to control the monitoring of an inflow stream for free water content, as performed by the system 500. The CPU processing system of the control device 502 may include one or more processors 506 coupled to a computer readable medium/memory 504 via a bus (not shown). The one or more processors 506 and the computer readable medium/memory 504 may communicate via a message passing interface (MPI) 536. In certain aspects the computer readable medium/memory 504 is configured to store instructions (e.g., computer executable code) that when executed by the one or more processors 506, cause the one or more processors to perform the method 600 described with respect to FIG. 6, or any aspect related to it. Reference to a processor performing a function of system 500 may include one or more processors performing that function of system 500.

In the depicted example, computer-readable medium/memory 504 stores code (e.g., executable instructions) for capturing 508, code for sensing 510, code for constructing 512, code for producing 514, code for applying 516, code for comparing 518, and code for obtaining 520. Processing of code 508-520 may cause the system 500 to perform the method 600 described with respect to FIG. 6, or any aspect related to it.

The one or more processors 506 include circuitry configured to implement (e.g., execute) the code stored in the computer-readable medium/memory 512, including circuitry for capturing 522, circuitry for sensing 524, and circuitry for constructing 526, circuitry for producing 528, circuitry for applying 530, circuitry for comparing 532, and circuitry for obtaining 534. Processing with circuitry 522-534 may cause the system 500 to perform the method 600 described with respect to FIG. 6, or any aspect related to it.

Various components of the system 500 may provide means for performing the method 600 described with respect to FIG. 6, or any aspect related to it.

The system 500 may include or be substantially coupled to a communication component 538. In the depicted example, the communication component 538 is an antenna capable of communicating with controllers similar to system 500 to perform the method 600 described with respect to FIG. 6, or any aspect related to it. In additional examples, the communication component 538 may be a bus or a wired connection.

FIG. 6 is a schematic flowchart of an example method 600 for monitoring liquid condensate and NGLs in an inflow stream. The method 600 may be performed by one or more CCD devices and/or by one or more processors, such as the processors of control device 502 of FIG. 5.

Method 600 begins at operation 602 with a CCD camera capturing one or more images of a flow stream, the flow stream comprising a water component and at least one of a liquid condensate component and a natural gas liquid component. In at least one embodiment, the at least one of the liquid condensate component and the natural gas liquid component comprise a hydrocarbon liquid mixture.

Method 600 continues to operation 604 with one or more processors sensing one or more bubble images captured within the one or more images of the flow stream, the one or more bubble images representing one or more water bubbles within the flow stream. In at least one embodiment, the sensing is refined based on a measured free water content value obtained via one or more coulometric KF titrations applied to one or more samples of the flow stream. In at least one embodiment, the measured free water content value is calculated based on a total water content value and a dissolved water content value.

Method 600 continues to operation 606 with one or more processors constructing one or more reference geometries based on the one or more bubble images, each of the one or more reference geometries having a parameter associated with each of the one or bubble images. In at least one embodiment, the one or more reference geometries include one or more circles over the one or more bubble images, each of the one or more circles having a diameter for each of the one or bubble images.

Method 600 continues to operation 608 with one or more processors, based on the parameter (e.g., diameter) for each of the one or bubble images, producing a predicted water content value for the flow stream. In at least one embodiment, producing the predicted water content value for the flow stream further comprises obtaining a total free water content volume value based on the diameter from each of the one or bubble images.

In at least one embodiment, the method 600 may include an operation by one or more processors to obtain the one or more images of the flow stream.

In at least one embodiment, the method 600 may include an operation by one or more processors to apply a machine learning engine to the one or more images of the flow stream, the machine learning engine comprising a training engine configured to train a machine learning model and an inference engine configured to apply the machine learning model. In at least one embodiment, the training engine is configured to train a machine learning model by: inputting a training data set, the training data set based on a set of measured free water content values obtained via one or more coulometric Karl Fischer (KF) titrations applied to one or more samples of the flow stream; comparing, to the training data set, an output of the training engine; and based on the comparing, adjusting one or more weights of the machine learning model. In at least one embodiment, the inference engine is configured to: identify the one or more bubble images within the one or more images; and correlate the one or more bubble images with one or more water bubbles within the flow stream. Application of a machine learning engine to the one or more bubble images may constitute one or more technical improvements over conventional liquid condensate and NGL image processing techniques. A machine learning engine applied in an iterative manner to images of free water images may allow fine-tuning of the machine learning engine parameters (e.g., layers, weights as described below). Accordingly, operations described with respect to method 600 may more accurately and more quickly predict free water content within an inflow stream when performed in connection with the machine learning engine described here. Additionally, application of the machine learning engine according to one or more embodiments described with respect to method 600 can perform accurate, real-time free water content prediction operations.

In at least one embodiment, the method 600 may include an operation by one or more processors to compare the predicted water content value to the measured free water content value; and obtaining an error rate based on the comparison.

In one aspect, method 600, or any aspect related to it, may be performed by a device, such as control device 502 of FIG. 5, which includes various components operable, configured to, or adapted to perform the method 600.

FIG. 6 is just one example of a method, and other methods including fewer, additional, or alternative operations are contemplated consistent with the disclosure.

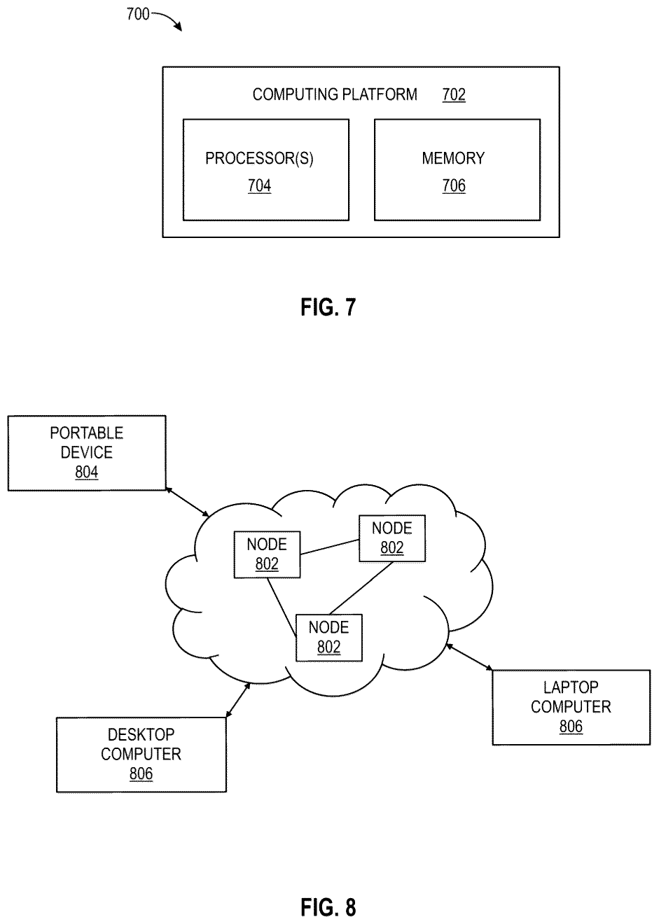

FIG. 7 is an example of a block diagram of a system 700. The system 700 can be implemented using one or more modules, shown in block form in the drawings. The one or more modules can be in software or hardware form, or a combination thereof. In some examples, the system 700 can be implemented as machine readable instructions for execution on one or more computing platforms 702 (referred to as a computing platform herein), as shown in FIG. 7. The computing platform 702 can include one or more computing devices selected from, for example, a desktop computer, a server, a controller, a blade, a mobile phone, a tablet, a laptop, a personal digital assistant (PDA), and the like.

The computing platform 707 can include a processor 704 and a memory 706. By way of example, the memory 706 can be implemented, for example, as a non-transitory computer storage medium, such as volatile memory (e.g., random access memory), non-volatile memory (e.g., a hard disk drive, a solid-state drive, a flash memory, or the like), or a combination thereof. The processor 704 can be implemented, for example, as one or more processor cores. The memory 706 can store machine-readable instructions that can be retrieved and executed by the processor 704 to implement the system 700. Each of the processor 704 and the memory 706 can be implemented on a similar or a different computing platform. The computing platform 702 can be implemented in a cloud computing environment (for example, as disclosed herein) and thus on a cloud infrastructure. In such a situation, features of the computing platform 702 can be representative of a single instance of hardware or multiple instances of hardware executing across the multiple of instances (e.g., distributed) of hardware (e.g., computers, routers, memory, processors, or a combination thereof). Alternatively, the computing platform 702 can be implemented on a single dedicated server or workstation.

Cloud computing is a model of service delivery for enabling convenient, on-demand network access to a shared pool of configurable computing resources (e.g., networks, network bandwidth, servers, processing, memory, storage, applications, virtual machines, and services) that can be rapidly provisioned and released with minimal management effort or interaction with a provider of the service. This cloud model may include at least five characteristics, at least three service models (e.g., software as a service (Saas, platform as a service (PaaS), and/or infrastructure as a service (IaaS)) and at least four deployment models (e.g., private cloud, community cloud, public cloud, and/or hybrid cloud). A cloud computing environment can be service oriented with a focus on statelessness, low coupling, modularity, and semantic interoperability.

FIG. 8 is an example of a cloud computing environment 800 that can be used for implementing one or more modules and/or systems in accordance with one or more examples, as disclosed herein. Thus, reference can be made to one or more examples of FIGS. 1-6 in the example of FIG. 8. As shown, cloud computing environment 800 can include one or more cloud computing nodes 802 with which local computing devices used by cloud consumers (or users), such as, for example, personal digital assistant (PDA), cellular, or portable device 804, a desktop computer 806, and/or a laptop computer 808, may communicate. The computing nodes 802 can communicate with one another. In some examples, the computing nodes 802 can be grouped (not shown) physically or virtually, in one or more networks, such as Private, Community, Public, or Hybrid clouds, or a combination thereof. This allows the cloud computing environment 800 to offer infrastructure, platforms and/or software as services for which a cloud consumer does not need to maintain resources on a local computing device. The devices 804-808, as shown in FIG. 8, are intended to be illustrative and that computing nodes 802 and cloud computing environment 800 can communicate with any type of computerized device over any type of network and/or network addressable connection (e.g., using a web browser). In some examples, the one or more computing nodes 802 are used for implementing one or more examples disclosed herein relating to root-source identification. Thus, in some examples, the one or more computing nodes can be used to implement modules, platforms, and/or systems, as disclosed herein.

In some examples, the cloud computing environment 800 can provide one or more functional abstraction layers. It is to be understood that the cloud computing environment 800 need not provide all of the one or more functional abstraction layers (and corresponding functions and/or components), as disclosed herein. For example, the cloud computing environment 800 can provide a hardware and software layer that can include hardware and software components. Examples of hardware components include: mainframes; RISC (Reduced Instruction Set Computer) architecture based servers; servers; blade servers; storage devices; and networks and networking components. In some embodiments, software components include network application server software and database software.

In some examples, the cloud computing environment 800 can provide a virtualization layer that provides an abstraction layer from which the following examples of virtual entities may be provided: virtual servers; virtual storage; virtual networks, including virtual private networks; virtual applications and operating systems; and virtual clients. In some examples, the cloud computing environment 800 can provide a management layer that can provide the functions described below. For example, the management layer can provide resource provisioning that can provide dynamic procurement of computing resources and other resources that are utilized to perform tasks within the cloud computing environment. The management layer can also provide metering and pricing to provide cost tracking as resources are utilized within the cloud computing environment 800, and billing or invoicing for consumption of these resources. In one example, these resources may include application software licenses. Security provides identity verification for cloud consumers and tasks, as well as protection for data and other resources. The management layer can also provide a user portal that provides access to the cloud computing environment 800 for consumers and system administrators. The management layer can also provide service level management, which can provide cloud computing resource allocation and management such that required service levels are met. Service Level Agreement (SLA) planning and fulfillment can also be provided to provide pre-arrangement for, and procurement of, cloud computing resources for which a future requirement is anticipated in accordance with an SLA.

In some examples, the cloud computing environment 800 can provide a workloads layer that provides examples of functionality for which the cloud computing environment 800 may be utilized. Examples of workloads and functions which may be provided from this layer include: mapping and navigation; software development and lifecycle management; virtual classroom education delivery; data analytics processing; and transaction processing. Various embodiments of the present disclosure can utilize the cloud computing environment 800.

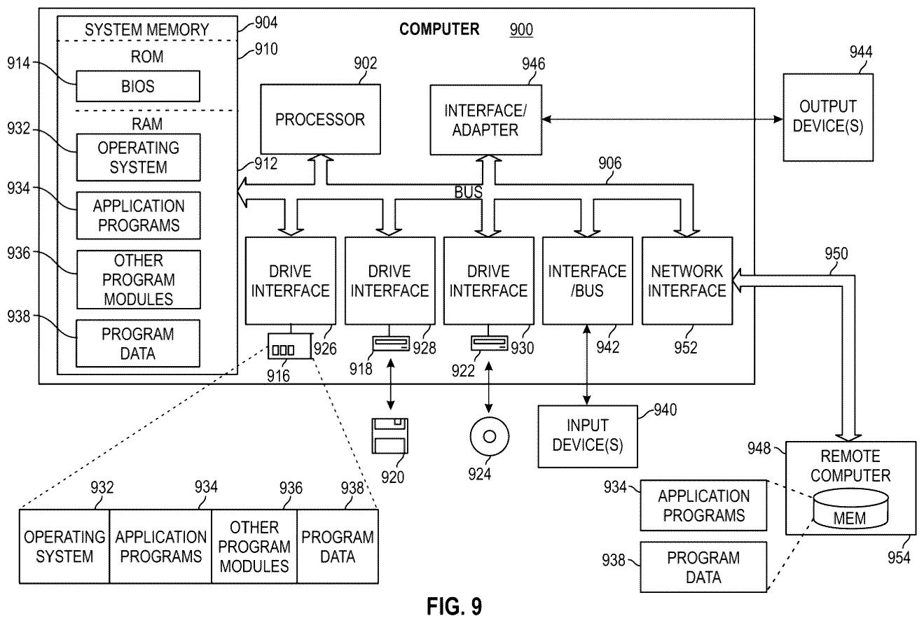

In view of the structural and functional features described above, example methods will be better appreciated with reference to FIG. 9. While, for purposes of simplicity of explanation, the example method of FIG. 9 are shown and described as executing serially, it is to be understood and appreciated that the present examples are not limited by the illustrated order, as some actions could in other examples occur in different orders, multiple times and/or concurrently from that shown and described herein. Moreover, it is not necessary that all described actions be performed to implement the methods, and conversely, some actions may be performed that are omitted from the description.

In view of the foregoing structural and functional description, those skilled in the art will appreciate that portions of the embodiments may be embodied as a method, data processing system, or computer program product. Accordingly, these portions of the present embodiments may take the form of an entirely hardware embodiment, an entirely software embodiment, or an embodiment combining software and hardware, such as shown and described with respect to the computer system of FIG. 9. Furthermore, portions of the embodiments may be a computer program product on a computer-readable storage medium having computer readable program code on the medium. Any non-transitory, tangible storage media possessing structure may be utilized including, but not limited to, static and dynamic storage devices, volatile and non-volatile memories, hard disks, optical storage devices, and magnetic storage devices, but excludes any medium that is not eligible for patent protection under 35 U.S.C. § 101 (such as a propagating electrical or electromagnetic signals per se). As an example and not by way of limitation, computer-readable storage media may include a semiconductor-based circuit or device or other IC (such, as for example, a field-programmable gate array (FPGA) or an ASIC), a hard disk, an HDD, a hybrid hard drive (HHD), an optical disc, an optical disc drive (ODD), a magneto-optical disc, a magneto-optical drive, a floppy disk, a floppy disk drive (FDD), magnetic tape, a holographic storage medium, a solid-state drive (SSD), a RAM-drive, a SECURE DIGITAL card, a SECURE DIGITAL drive, or another suitable computer-readable storage medium or a combination of two or more of these, where appropriate. A computer-readable non-transitory storage medium may be volatile, nonvolatile, or a combination of volatile and non-volatile, as appropriate.

Certain embodiments have also been described herein with reference to block illustrations of methods, systems, and computer program products. It will be understood that blocks and/or combinations of blocks in the illustrations, as well as methods or steps or acts or processes described herein, can be implemented by a computer program comprising a routine of set instructions stored in a machine-readable storage medium as described herein. These instructions may be provided to one or more processors of a general purpose computer, special purpose computer, or other programmable data processing apparatus (or a combination of devices and circuits) to produce a machine, such that the instructions of the machine, when executed by the processor, implement the functions specified in the block or blocks, or in the acts, steps, methods and processes described herein.

These processor-executable instructions may also be stored in computer-readable memory that can direct a computer or other programmable data processing apparatus to function in a particular manner, such that the instructions stored in the computer-readable memory result in an article of manufacture including instructions which implement the function specified. The computer program instructions may also be loaded onto a computer or other programmable data processing apparatus to cause a series of operational steps to be performed on the computer or other programmable apparatus to realize a computer implemented process such that the instructions which execute on the computer or other programmable apparatus provide steps for implementing the functions specified in flowchart blocks that may be described herein.

In this regard, FIG. 9 illustrates one example of a computer system 900 that can be employed to execute one or more embodiments of the present disclosure. Computer system 900 can be implemented on one or more general purpose networked computer systems, embedded computer systems, routers, switches, server devices, client devices, various intermediate devices/nodes or standalone computer systems. Additionally, computer system 900 can be implemented on various mobile clients such as, for example, a personal digital assistant (PDA), laptop computer, pager, and the like, provided it includes sufficient processing capabilities.

Computer system 900 includes processing unit 902, system memory 904, and system bus 906 that couples various system components, including the system memory 904, to processing unit 902. System memory 904 can include volatile (e.g. RAM, DRAM, SDRAM, Double Data Rate (DDR) RAM, etc.) and non-volatile (e.g. Flash, NAND, etc.) memory. Dual microprocessors and other multi-processor architectures also can be used as processing unit 902. System bus 906 may be any of several types of bus structure including a memory bus or memory controller, a peripheral bus, and a local bus using any of a variety of bus architectures. System memory 904 includes read only memory (ROM) 910 and random access memory (RAM) 912. A basic input/output system (BIOS) 914 can reside in ROM 910 containing the basic routines that help to transfer information among elements within computer system 900.

Computer system 900 can include a hard disk drive 916, magnetic disk drive 918, e.g., to read from or write to removable disk 920, and an optical disk drive 922, e.g., for reading CD-ROM disk 924 or to read from or write to other optical media. Hard disk drive 916, magnetic disk drive 918, and optical disk drive 922 are connected to system bus 906 by a hard disk drive interface 926, a magnetic disk drive interface 928, and an optical drive interface 930, respectively. The drives and associated computer-readable media provide nonvolatile storage of data, data structures, and computer-executable instructions for computer system 900. Although the description of computer-readable media above refers to a hard disk, a removable magnetic disk and a CD, other types of media that are readable by a computer, such as magnetic cassettes, flash memory cards, digital video disks and the like, in a variety of forms, may also be used in the operating environment; further, any such media may contain computer-executable instructions for implementing one or more parts of embodiments shown and described herein.

A number of program modules may be stored in drives and RAM 910, including operating system 932, one or more application programs 934, other program modules 936, and program data 938.

A user may enter commands and information into computer system 900 through one or more input devices 940, such as a pointing device (e.g., a mouse, touch screen), keyboard, microphone, joystick, game pad, scanner, and the like. These and other input devices 940 are often connected to processing unit 902 through a corresponding port interface 942 that is coupled to the system bus, but may be connected by other interfaces, such as a parallel port, serial port, or universal serial bus (USB). One or more output devices 944 (e.g., display, a monitor, printer, projector, or other type of displaying device) is also connected to system bus 906 via interface 946, such as a video adapter.

Computer system 900 may operate in a networked environment using logical connections to one or more remote computers, such as remote computer 948. Remote computer 948 may be a workstation, computer system, router, peer device, or other common network node, and typically includes many or all the elements described relative to computer system 900. The logical connections, schematically indicated at 950, can include a local area network (LAN) and/or a wide area network (WAN), or a combination of these, and can be in a cloud-type architecture, for example configured as private clouds, public clouds, hybrid clouds, and multi-clouds. When used in a LAN networking environment, computer system 900 can be connected to the local network through a network interface or adapter 952. When used in a WAN networking environment, computer system 900 can include a modem, or can be connected to a communications server on the LAN. The modem, which may be internal or external, can be connected to system bus 906 via an appropriate port interface. In a networked environment, application programs 934 or program data 938 depicted relative to computer system 900, or portions thereof, may be stored in a remote memory storage device 954.

Although this disclosure includes a detailed description on a computing platform and/or computer, implementation of the teachings recited herein are not limited to only such computing platforms. Rather, embodiments of the present disclosure are capable of being implemented in conjunction with any other type of computing environment now known or later developed.

Implementation examples are described in the following numbered clauses:

Aspect 1: A method for detecting water content, including capturing one or more images of a flow stream, the flow stream comprising a water component and at least one of a liquid condensate component and a natural gas liquid component; sensing one or more bubble images captured within the one or more images of the flow stream, the one or more bubble images representing one or more water bubbles within the flow stream, wherein the sensing is refined based on a measured free water content value and the measured free water content value is based on a total water content value and a dissolved water content value; constructing one or more reference geometries based on the one or more bubble images, each of the one or more reference geometries having a parameter associated with each of the one or bubble images; and based on the parameter for each of the one or bubble images, producing a predicted water content value for the flow stream.

Aspect 2: The method of aspect 1, wherein: a charged couple device (CCD) captures the one or more images of the flow stream; and a control device obtains the one or more images of the flow stream.

Aspect 3: The method of any one of aspects 1 through 2, wherein the at least one of the liquid condensate component and the natural gas liquid component comprise a hydrocarbon liquid mixture.

Aspect 4: The method of any one of aspects 1 through 3, wherein the method further includes applying a machine learning engine to the one or more images of the flow stream, the machine learning engine comprising a training engine configured to train a machine learning model and an inference engine configured to apply the machine learning model.

Aspect 5: The method of any one of aspects 1 through 4, wherein the training engine is configured to train a machine learning model by inputting a training data set, the training data set based on a set of measured free water content values obtained via one or more coulometric Karl Fischer (KF) titrations applied to one or more samples of the flow stream; comparing, to the training data set, an output of the training engine; and based on the comparing, adjusting one or more weights of the machine learning model.

Aspect 6: The method of any one of aspects 4 through 5, wherein the inference engine is configured to: identify the one or more bubble images within the one or more images; and correlate the one or more bubble images with one or more water bubbles within the flow stream.

Aspect 7: The method of any one of aspects 1 through 6, wherein producing the predicted water content value for the flow stream further comprises obtaining a total free water content volume value based on the diameter from each of the one or bubble images.

Aspect 8: The method of any one of aspects 1 through 7, wherein the measured free water content value is obtained via one or more coulometric Karl Fischer (KF) titrations applied to one or more samples of the flow stream.

Aspect 9: The method of any one of aspects 1 through 8, further including comparing the predicted water content value to the measured free water content value; and obtaining an error rate based on the comparison.

Aspect 10: An apparatus or device including a memory comprising executable instructions, and a processor configured to execute the executable instructions and cause the apparatus to perform a method in accordance with any one of aspects 1-9.

Aspect 11: An apparatus or device, including means for performing a method in accordance with any one of aspects 1-9.

Aspect 12: A non-transitory computer-readable medium including executable instructions that, when executed by a processor of an apparatus, cause the apparatus to perform a method in accordance with any one of aspects 1-9.

Aspect 12: A computer program product embodied on a computer-readable storage medium comprising code for performing a method in accordance with any one of aspects 1-9.

The present invention may be a system, a method, and/or a computer program product at any possible technical detail level of integration. The computer program product may include a computer readable storage medium (or media) having computer readable program instructions thereon for causing a processor to carry out aspects of the present invention. The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware based systems that perform the specified functions or acts or carry out combinations of special purpose hardware and computer instructions.

The terminology used herein is for the purpose of describing particular embodiments only and is not intended to be limiting of the invention. As used herein, for example, the singular forms “a,” “an,” and “the” are intended to include the plural forms as well, unless the context clearly indicates otherwise. It will be further understood that the terms “contains”, “containing”, “includes”, “including,” “comprises”, and/or “comprising,” and variations thereof, when used in this specification, specify the presence of stated features, integers, steps, operations, elements, and/or components, but do not preclude the presence or addition of one or more other features, integers, steps, operations, elements, components, and/or groups thereof.

Terms of orientation used herein are merely for purposes of convention and referencing and are not to be construed as limiting. However, it is recognized these terms could be used with reference to an operator or user. Accordingly, no limitations are implied or to be inferred. In addition, the use of ordinal numbers (e.g., first, second, third, etc.) is for distinction and not counting. For example, the use of “third” does not imply there must be a corresponding “first” or “second.” Also, if used herein, the terms “coupled” or “coupled to” or “connected” or “connected to” or “attached” or “attached to” may indicate establishing either a direct or indirect connection, and is not limited to either unless expressly referenced as such. Furthermore, to the extent that the terms “includes,” “has,” “possesses,” and the like are used in the detailed description, claims, appendices and drawings such terms are intended to be inclusive in a manner similar to the term “comprising” as “comprising” is interpreted when employed as a transitional word in a claim. The term “based on” means “based at least in part on.” The terms “about” and “approximately” can be used to include any numerical value that can vary without changing the basic function of that value. When used with a range, “about” and “approximately” also disclose the range defined by the absolute values of the two endpoints, e.g. “about 2 to about 4” also discloses the range “from 2 to 4.” Generally, the terms “about” and “approximately” may refer to plus or minus 5-10% of the indicated number.

While the disclosure has described several exemplary embodiments, it will be understood by those skilled in the art that various changes can be made, and equivalents can be substituted for elements thereof, without departing from the spirit and scope of the invention. In addition, many modifications will be appreciated by those skilled in the art to adapt a particular instrument, situation, or material to embodiments of the disclosure without departing from the essential scope thereof. Therefore, it is intended that the invention not be limited to the particular embodiments disclosed, or to the best mode contemplated for carrying out this invention, but that the invention will include all embodiments falling within the scope of the appended claims. Moreover, reference in the appended claims to an apparatus or system or a component of an apparatus or system being adapted to, arranged to, capable of, configured to, enabled to, operable to, or operative to perform a particular function encompasses that apparatus, system, or component, whether or not it or that particular function is activated, turned on, or unlocked, as long as that apparatus, system, or component is so adapted, arranged, capable, configured, enabled, operable, or operative.

1. A method for detecting water content, comprising:
capturing one or more images of a flow stream, the flow stream comprising a water component and at least one of a liquid condensate component and a natural gas liquid component;
sensing one or more bubble images captured within the one or more images of the flow stream, the one or more bubble images representing one or more water bubbles within the flow stream, wherein the sensing is refined based on a measured free water content value and the measured free water content value is based on a total water content value and a dissolved water content value;
constructing one or more reference geometries based on the one or more bubble images, each of the one or more reference geometries having a parameter associated with each of the one or bubble images; and
based on the parameter for each of the one or bubble images, producing a predicted water content value for the flow stream.

2. The method of claim 1, wherein:
a charged couple device (CCD) captures the one or more images of the flow stream; and
a control device obtains the one or more images of the flow stream.

3. The method of claim 1, wherein the at least one of the liquid condensate component and the natural gas liquid component comprise a hydrocarbon liquid mixture.

4. The method of claim 1, wherein the method further comprises:
applying a machine learning engine to the one or more images of the flow stream, the machine learning engine comprising a training engine configured to train a machine learning model and an inference engine configured to apply the machine learning model.

5. The method of claim 4, wherein the training engine is configured to train a machine learning model by:
inputting a training data set, the training data set based on a set of measured free water content values obtained via one or more coulometric Karl Fischer (KF) titrations applied to one or more samples of the flow stream;
comparing, to the training data set, an output of the training engine; and
based on the comparing, adjusting one or more weights of the machine learning model.

6. The method of claim 4, wherein the inference engine is configured to:
identify the one or more bubble images within the one or more images; and
correlate the one or more bubble images with one or more water bubbles within the flow stream.

7. The method of claim 1, wherein producing the predicted water content value for the flow stream further comprises obtaining a total free water content volume value based on the diameter from each of the one or bubble images.

8. The method of claim 1, wherein the measured free water content value is obtained via one or more coulometric Karl Fischer (KF) titrations applied to one or more samples of the flow stream.

9. The method of claim 1, further comprising:
comparing the predicted water content value to the measured free water content value; and
obtaining an error rate based on the comparison.

10. A monitoring system for detecting water content, comprising:
a charged couple device (CCD) configured to capture one or more images of a flow stream, the flow stream comprising a water component and at least one of a liquid condensate component and a natural gas liquid component; and
a control device having a memory coupled to one or more processors, the one or more processors configured to cause the control device to:
sense one or more bubble images captured within the one or more images of the flow stream, the one or more bubble images representing one or more water bubbles within the flow stream, wherein the sensing is refined based on a measured free water content value and the measured free water content value is based on a total water content value and a dissolved water content value;
construct one or more reference geometries based on the one or more bubble images, each of the one or more reference geometries having a parameter associated with each of the one or bubble images; and
based on the parameter for each of the one or bubble images, produce a predicted water content value for the flow stream.

11. The monitoring system of claim 10, wherein the at least one of the liquid condensate component and the natural gas liquid component comprise a hydrocarbon liquid mixture.

12. The monitoring system of claim 1, wherein the one or more processors are further configured to cause the control device to:
apply a machine learning engine to the one or more images of the flow stream, the machine learning engine comprising a training engine configured to train a machine learning model and an inference engine configured to apply the machine learning model.

13. The monitoring system of claim 12, wherein the training engine is configured to train a machine learning model by:
inputting a training data set, the training data set based on a set of measured free water content values obtained via one or more coulometric Karl Fischer (KF) titrations applied to one or more samples of the flow stream;
comparing, to the training data set, an output of the training engine; and
based on the comparing, adjusting one or more weights of the machine learning model.

14. The monitoring system of claim 12, wherein the inference engine is configured to:
identify the one or more bubble images within the one or more images; and
correlate the one or more bubble images with one or more water bubbles within the flow stream.

15. The monitoring system of claim 10, wherein producing the predicted water content value for the flow stream further comprises obtaining a total free water content volume value based on the diameter from each of the one or bubble images.

16. The monitoring system of claim 10, wherein the measured free water content value is obtained via one or more coulometric Karl Fischer (KF) titrations applied to one or more samples of the flow stream.

17. The monitoring system of claim 10, further comprising:
comparing the predicted water content value to the measured free water content value; and
obtaining an error rate based on the comparison.

18. A non-transitory computer readable medium for detecting water content, the non-transitory computer readable medium comprising a memory coupled to one or more processors, the one or more processors configured to:
capture one or more images of a flow stream, the flow stream comprising a water component and at least one of a liquid condensate component and a natural gas liquid component;
sense one or more bubble images captured within the one or more images of the flow stream, the one or more bubble images representing one or more water bubbles within the flow stream, wherein the sensing is refined based on a measured free water content value and the measured free water content value is based on a total water content value and a dissolved water content value;
construct one or more reference geometries based on the one or more bubble images, each of the one or more reference geometries having a parameter associated with each of the one or bubble images; and
based on the parameter for each of the one or bubble images, produce a predicted water content value for the flow stream.
